(19) United States
(12) Patent Application Publication
(10) Pub. No.: US 2017/0305855 A1
(43) Pub. Date: Oct. 26, 2017

(54) STERICALLY HINDERED AMINE AND OXYALKYL AMINE LIGHT STABILIZERS

(71) Applicant: 3M INNOVATIVE PROPERTIES COMPANY, St. Paul, MN (US)

(72) Inventors: Thomas P. Klun, Lakeland, MN (US); Mark A. Roehrig, Stillwater, MN (US); Joseph C. Spagnola, Woodbury, MN (US); Alan K. Nachtigal, Maplewood, MN (US); Charles J. Hoy, Maple Plain, MN (US); Richard J. Pokorny, Maplewood, MN (US); William J. Hunt, Afton, MN (US); Jason T. Petrin, Woodbury, MN (US); Paul B. Armstrong, St. Paul, MN (US); Suresh S. Iyer, Woodbury, MN (US)

(21) Appl. No.: 15/531,767

(22) PCT Filed: Dec. 11, 2015
(86) PCT No.: PCT/US15/65211

§ 371 Date: May 31, 2017

Related U.S. Application Data
(60) Provisional application No. 62/095,523, filed on Dec. 22, 2014.

(51) Int. Cl. C07D 211/36; C07D 211/94; C07D 211/58; C08F 220/58; C07D 211/44

(52) U.S. Cl. CPC C07D 211/36; C08F 220/58; C07D 211/44; C07D 211/58; C07D 211/94

(57) ABSTRACT

Compounds having hindered amine and oxyalkyl amine light stabilizers can mitigate the adverse effects of actinic radiation, such as visible and ultraviolet light, on polymers and copolymers. Polymers and copolymers derived from such compounds. Articles, such as coated articles and molded articles, containing such polymers or compounds.

### CROSS REFERENCE TO RELATED APPLICATION

This application claims the benefit of U.S. Provisional Patent Application No. 62/095,523, filed Dec. 22, 2014, the disclosure of which is incorporated by reference herein in its entirety.

### TECHNICAL FIELD

The present disclosure relates to sterically hindered alkyl amine and sterically hindered oxyalkyl amine compounds.

### BACKGROUND

Compounds containing sterically hindered alkyl amines or sterically hindered oxyalkyl amines, and particularly the moiety

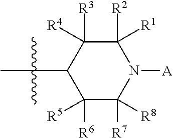

wherein R¹ is alkyl, R² is alkyl, R³ is H or alkyl, R⁴ is H or alkyl, R⁵ is H or alkyl, R⁶ is H or alkyl, R⁷ is alkyl, and R⁸ is alkyl is known in the art. When A is alkyl, such compounds are known as hindered amine light stabilizers, or HALS; when A is oxyalkyl, such compounds are known as NORHALS.

The utility of HALS and NORHALS as radical scavengers and polymer stabilizers and is well recognized in the art, and is described in, for example, the Journal of Macromolecular Science Part A, 35:7, 1327-36 (1998) and The Journal of Macromolecular Science Part A, 38:2, 137-58 (2001), as well as in JP 2001270859, U.S. Pat. No. 4,983,737 (Grant), and U.S. Pat. No. 5,442,071 (Grant). Such compounds are known to mitigate the adverse effects of actinic radiation, such as visible and ultraviolet light.

### SUMMARY

A compound can have the structure of Formula (I):

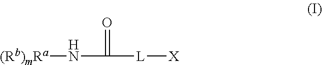

wherein:

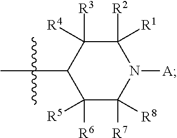

### X is

R¹ is alkyl;

R² is alkyl;

R³ is H or alkyl;

R⁴ is H or alkyl;

R⁵ is H or alkyl;

R⁶ is H or alkyl;

R⁷ is alkyl;

R⁸ is alkyl;

A is alkyl or oxyalkyl;

L is O or NR′;

R′ is H or C₁ to C₄ alkyl;

m is 1-6;

R^{a} is a connecting group having a valence of m+1; and

R^{b} is an (alkyl)acrylolyoxy functional group of the formula OC(O)C(R^{d})═CH₂ where R^{d} is alkyl or H.

### DETAILED DESCRIPTION

Throughout this disclosure, singular forms such as “a,” “an,” and “the” are often used for convenience; however, it should be understood that the singular forms are meant to include the plural unless the singular alone is explicitly specified or is clearly indicated by the context.

Some terms used in this application have special meanings, as defined herein. All other terms will be known to the skilled artisan, and are to be afforded the meaning that a person of skill in the art at the time of the invention would have given them.

“Independently,” when used in reference to the identity of one or more variable elements, means that each occurrence of any of the variable elements may have the same or different identity, within the specified limitations, regardless of the identity of any other occurrence of the reference element. Thus, if there are two occurrences of element “X,” and element X can be independently selected from identity Y or identity Z, each of the two occurrences of X can be either Y or Z, in any combination (e.g., YY, YZ, ZY, or ZZ).

“Alkyl” refers to an aliphatic hydrocarbon radical. Many alkyl groups are from C₁ to C₃₀. Some alkyl groups can be C₁ or greater, such as C₂ or greater, C₄ or greater, C₆ or greater, or C₈ or greater. Some alkyl groups can be C₂₂ or smaller, C₁₆ or smaller, C₁₂ or smaller, C₈ or smaller, or C₄ or smaller. Unless otherwise indicated, any alkyl group can independently be linear, branched, cyclic, or a combination thereof (e.g., a cyclic alkyl can also have a linear or branched component.) Exemplary alkyl groups include methyl, ethyl, propyl, isopropyl, n-butyl, t-butyl, sec-butyl, iso-butyl, 2-ethyl hexyl, iso-octyl, n-octyl, dodecyl, hexadecyl, behenyl, and the like.

“Oxyalkyl” refers to a monovalent radical having the formula O-alkyl, which can be referred to as an alkoxy group. The alkyl portion of the oxyalkyl can be any alkyl, such as those discussed above with reference to the definition of the term alkyl. Oxyalkyl can be written using standard nomenclature to indicate the number of carbon atoms in the alkyl portion of the oxyalkyl. For example, oxymethyl is an oxyalkyl wherein the alkyl portion has one carbon, oxyethyl is an oxyalkyl wherein the alkyl portion has two carbons, etc. Oxyoctyl is an exemplary oxyalkyl that is often used in the compounds described herein.

“Alkylene” refers to an aliphatic hydrocarbon diradical (i.e., divalent radical). Many alkylene diradicals are from C₁ to C₃₀. Alkylene diradicals can be C₁ or greater, C₂ or greater, C₃ or greater, C₄ or greater, C₆ or greater, or C₈ or greater. Alkylene diradicals can be C₂₂ or smaller, C₁₆ or smaller, C₁₂ or smaller, C₁₀ or smaller, or C₈ or smaller. Unless otherwise indicated, any alkylene can be linear, branched or cyclic or a combination thereof (e.g., having both a cyclic component and a linear component.) Exemplary alkylene groups include methylene, ethylene, propyl, isopropylene, n-butylene, t-butylene, sec-butylene, iso-butylene, 2-ethylhexylene, iso-octylene, dodecylene, hexadecylene, behenylene, and the like.

“Isocyanate” refers to a molecule comprising at least one isocyanato group, which is a —NCO radical.

A polymer or copolymer is “derived from” a reference compound when the backbone of the polymer or copolymer contains a polymerized form of the reference compound, either by itself or in combination with other polymerized monomers.

A “hydrocarbon polyradical” as used herein is an aliphatic multivalent radical having a valence of at least three and containing only carbon and hydrogen atoms. Hydrocarbon polyradicals can be from C₁ to C₃₀. Many are C₁ or greater, C₂ or greater, C₃ or greater, C₄ or greater, C₆ or greater, or C₈ or greater. Hydrocarbon polyradicals can be C₃₀ or smaller, C₂₂ or smaller, C₁₆ or smaller, C₁₂ or smaller, C₁₀ or smaller, or C₈ or smaller. In many embodiments, the polyradicals are divalent or trivalent.

Compounds of Formula (I) can have an L that is O or NR′ with R′ being H or C₁ to C₄ alkyl. When L is O, the compound of Formula (I) is a compound of Formula (II). When L is NR′, the compound of Formula (I) is a compound of Formula (IIa).

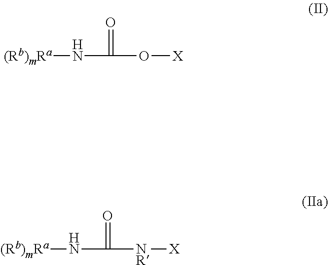

Compounds of Formulas (I) can be synthesized from compounds of Formula (III).

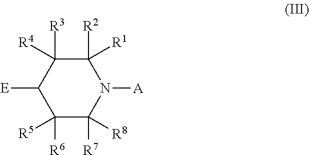

In the compound of Formula (III), R¹ through R⁸, and A have the same meaning as in the compound of Formula (I), and E is OH or NHR′, wherein R′ has the same meaning as in the compound of Formula (I).

In any compound of Formula (III), R¹, R², R⁷, and R⁸ can be independently any suitable alkyl. R¹, R², R⁷, and R⁸ can be the same or different. Typical alkyls for any of R¹, R², R⁷, and R⁸ include C₁ or greater, C₂ or greater, C₃ or greater, C₄ or greater, C₆ or greater, C₈ or greater, or C₁₂ or greater. Other typical alkyls that can be used as one or more of R¹, R², R⁷, and R⁸ include C₁₆ or less, C₁₂ or less, C₈ or less, C₆ or less, C₄ or less, C₃ or less, or C₂ or less. In many cases, each of R¹, R², R⁷, and R⁸ are methyl.

R³, R⁴, R⁵, and R⁶ can be independently H or alkyl. When one or more of R³, R⁴, R⁵, and R⁶ is alkyl, the alkyl is typically C₁ or greater, C₂ or greater, C₃ or greater, C₄ or greater, C₆ or greater, C₈ or greater, or C₁₂ or greater. Such alkyl is often C₁₆ or less, C₁₂ or less, C₈ or less, C₆ or less, C₄ or less, C₃ or less, or C₂ or less. In many cases, one or more of R³, R⁴, R⁵, and R⁶ is H. Most commonly, each of R³, R⁴, R⁵, and R⁶ are H.

The identity of each of R¹ through R⁸ in a compound of Formula (III) is carried over into compounds of Formula (I) that are synthesized from that compound of Formula (III). Thus, the identity of each of R¹ through R⁸ in any compound of Formula (I) will depend on, and be the same as, the identity of the R¹ through R⁸ in the compound or compounds of Formula (III) used as a starting material.

In some cases, E in the compound of Formula (III) is hydroxy. When such compound is employed as a starting material, the resulting compound of Formula (I) or Formula (II) will have an L that is O. A can be either alkyl or oxyalkyl. When A is alkyl, then the compound of Formula (III) is a compound of, for example, Formula (IIIa). When A is oxyalkyl, then the compound of Formula (III) is a compound of, for example, Formula (IIIb).

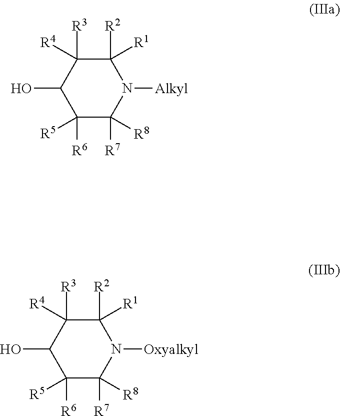

The alkyl in the compound of Formula (IIIa) can be any suitable alkyl. The alkyl can be linear, branched, cyclic, or a combination thereof (e.g., a cyclic alkyl that also has a linear component). Typical alkyls are C₁ or greater, C₂ or greater, C₃ or greater, C₄ or greater, C₆ or greater, C₈ or greater, or C₁₂ or greater. Many alkyls are C₁₆ or less, C₁₂ or less, C₈ or less, C₆ or less, C₄ or less, C₃ or less, or C₂ or less. In many cases, the alkyl is C₁ to C₄ alkyl. Methyl is most common.

Most commonly compounds of Formula (IIIa) feature R¹, R², R⁷, and R⁸ that are methyl, R³, R⁴, R⁵, and R⁶ that are H. In such cases, the compound of Formula (III) is a compound of Formula (IIIa1). The alkyl, which is connected to the nitrogen in the ring, in the compound of Formulas (IIIa) and (IIIa1) is most often methyl. In such cases, the compound of Formula (IIIa1) is a compound of Formula (IIIa2).

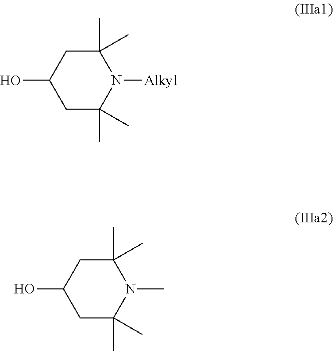

Most commonly compounds of Formula (IIIb) feature R¹, R², R⁷, and R⁸ that are methyl, and R³, R⁴, R⁵, and R⁶ that are H. In such cases, the compound of Formula (IIIb) is a compound of Formula (IIIb1).

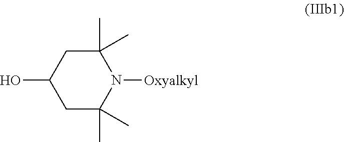

The oxyalkyl in the compound of Formula (IIIb) or (IIIb1), which is connected to the nitrogen in the ring, can be any suitable oxyalkyl. The oxyalkyl can be linear, branched, cyclic, or a combination thereof (e.g., a cyclic oxyalkyl can also have a linear component). Typical oxyalkyls are C₁ or greater, C₂ or greater, C₃ or greater, C₄ or greater, C₆ or greater, C₈ or greater, C₁₂ or greater, C₁₆ or greater, or C₂₂ or greater. Many oxyalkyls are C₂₆ or less, C₂₂ or less, C₁₈ or less, C₁₆ or less, C₁₂ or less, C₈ or less, C₆ or less, C₄ or less, C₃ or less, or C₂ or less. C₈ oxyalkyl is often used. In most cases, compounds of Formula (IIIb) or (IIIb1) contain a mixture of linear and branched isomers of the oxyalkyl group. This effect has been noted in documents that describe the preparation of such compounds, such as Schoening et al. (J. Org. Chem., 2009, 74, 1567-1573), U.S. Pat. Nos. 4,983,737, 5,286,865, 5,442,071 and US2010/0249401. Of the C₈ isomers, which are collectively known as oxyoctyl, branched isomers tend to occur more often than the linear isomer. When the oxyalkyl in the compound of Formula (IIIb1) is oxyoctyl, the compound of Formula (IIIb1) is a compound of Formula (IIIb2).

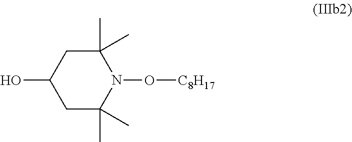

In other cases, E in the compound Formula (III) can be NHR′. When such compounds are employed as starting materials, the resulting compounds of Formula (I) or (II) will have L that is NR′. A can be alkyl or oxyalkyl. When A is alkyl, the compound of Formula (III) is a compound of Formula (IV). When A is oxyalkyl, the compound of Formula (III) is a compound of Formula (IVa).

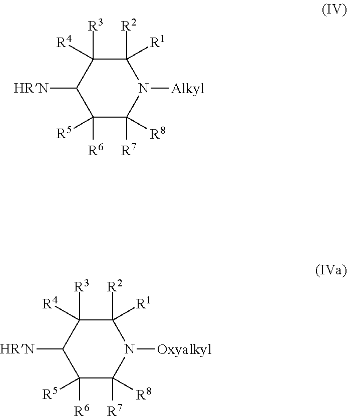

In compounds of Formula (IV) and (IVa), the identity of each of R¹ through R⁸ is the same as in the compound of Formula (III). Most commonly, compounds of Formula (IV) feature R¹, R², R⁷, and R⁸ that are methyl, and R³, R⁴, R⁵, and R⁶ that are H. In such cases, the compound of Formula (IV) is a compound of Formula (IV1).

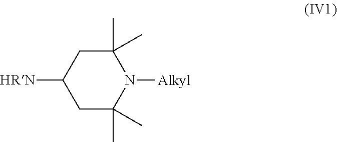

R′ in the compound of Formula (IV) or (IV1) can be H or any C₁ to C₄ alkyl. When R′ is alkyl, methyl and ethyl are most common. Typically, R′ is H, in which case the compound of Formula (IV1) is a compound of Formula (IV2).

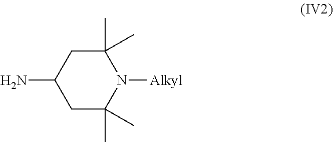

The alkyl, which is connected to the nitrogen in the ring, in the compound of Formula (IV), (IV1), or (IV2) can be any suitable alkyl, such as those discussed above with respect to the compound of Formulas (IIIa). Methyl is most common, in which case the compound of Formula (IV2) is a compound of Formula (IV3).

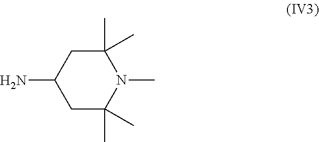

In the compound of Formula (IVa), the identity of each of R¹ through R⁸ is the same as in the compound of Formula (III). Most commonly, compounds of Formula (IVa) feature R¹, R², R⁷, and R⁸ that are methyl, R³, R⁴, R⁵, and R⁶ that are H. In such cases, the compound of Formula (IVa) is a compound of Formula (IVa1).

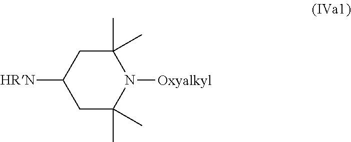

R′ in the compound of Formula (IVa) or (IVa1) can be H or any C₁ to C₄ alkyl. When R′ is alkyl, methyl and ethyl are most common. Typically, R′ is H, in which case the compound of Formula (IVa1) is a compound of Formula (IVa2)

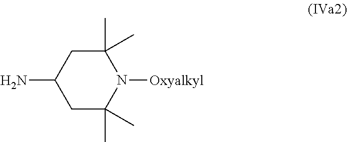

In the compounds of Formulas (IVa), (IVa1), and (IVa2) the oxyalkyl, which is connected to the nitrogen in the ring, can be any suitable oxyalkyl, such as those discussed above with respect to the compound of Formula (IIIb). Oxyoctyl is most common, in which case the compound of Formula (IVa2) is a compound of Formula (IVa3)

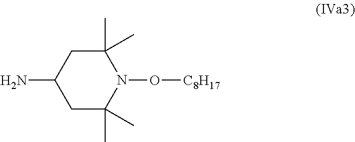

The various compounds of Formula (III) discussed herein can be used in the synthesis of compounds of Formulas (I), (II), or (IIa). Those compounds featuring and E that is OH are useful in preparing compounds of Formula (I) or (II), whereas those featuring an E that is NHR′ are useful for preparing compounds of Formula (I) or (IIa).

For example, compounds of Formula (IIIa) can be used as starting materials for compounds of Formula (I) wherein A is alkyl and L is O. Typically, compounds of Formula (IIIa2) are used for this purpose. Exemplary compounds of Formula (IIIa), (IIIa1), and (IIIa2) can be obtained from TCI America (OR, USA), for example, under the trade designation PMHP.

As another example, compounds of Formula (IIIb), (IIIb1), and (IIIb2) can be used as starting materials for compounds of Formula (I) wherein A is oxyalkyl and L is O. Compounds of Formula (IIIb) are sometimes known as alkylated N-oxyalkyl 4-hydroxy piperidines, and can be prepared from commercially available bis(alkyated N-oxyalkyl-4-piperidyl) esters of alkylene diacids as shown in Reaction Scheme 1. Exemplary bis(alkylated N-oxyalkyl-4-piperidyl) esters of alkylene diacids can be obtained from BASF (NJ, USA), for example, under the trade designation TINUVIN 123.

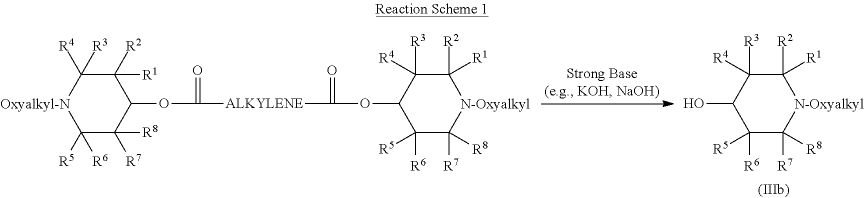

As shown in Reaction Scheme 1, treating a bis(alkylated N-oxyalkyl-4-piperidyl) ester of alkylene diacids with a strong Arrhenius base, for example an alkali metal hydroxide such as potassium hydroxide or sodium hydroxide, hydrolyzes the esters to form an alkylated N-oxyalkyl 4-hydroxy piperidine. This reaction can take place under any suitable conditions for hydrolyzing diacids. The reaction often takes place in the presence of one or more inert diluents. The one or more inert diluents are typically used to dissolve or disperse the strong Arrhenius base, the bis(alkylated N-oxyalkyl-4-piperidyl) esters of alkylene diacids, or both. Typical inert diluents include alcohols, such as methanol, ethanol, or isopropanol. The reaction can be promoted by heating. When one or more alcohols are used as the inert diluents, heating can involve refluxing the one or more alcohols. The starting material of Reaction Scheme 1 is often a bis(2,2,6,6-tetramethyl-N-oxyalkyl-4-piperidyl) ester, in which case the product of Reaction Scheme 1 is the compound of Formula (IIIb2).

Compounds of Formula (IV), including compounds of Formula (IV1), (IV2), and (IV3), can be used as starting materials for compounds of Formula (I) wherein A is alkyl and L is NR′. Compounds of Formula (IVa), including compounds of Formulas (IVa1), (IVa2), and (IVa3), can be used as starting materials for compounds of Formula (I) wherein A is oxyalkyl and L is NR′.

Compounds of Formula (IV) and (IVa) wherein R′ is H are compounds of Formula (V) and (Va), respectively. Such compounds can be synthesized from compounds of Formula (IIIa) or (IIIb), respectively, as shown in Reaction Scheme 2 and Reaction Scheme 3. First, compounds of Formulas (IIIa) or (IIIb) can be converted to ketone intermediates of, respectively, Formula (IIIc) or (IIId) by Swern oxidation of the hydroxy group with oxalyl chloride and dimethyl sulfoxide (DMSO) followed by quenching with triethylamine. The ketone intermediates of Formula (IIIc) or (IIId) can then be converted to compounds of Formula (IV) or (IVa), respectively, by reductive amination. Reductive amination can be accomplished by any suitable procedure, such as treatment with sodium cyanoborohydride and ammonia or an amine, which is typically a protonated ammonia (an ammonium salt such as ammonium acetate) or a protonated amine.

The nature of the amine used in the reductive amination reaction determines the identity of R′ in the compound of Formula (IV) or (IVa). Thus, if ammonium is used, as in Reaction Scheme 2, R′ in the resulting compound is H.

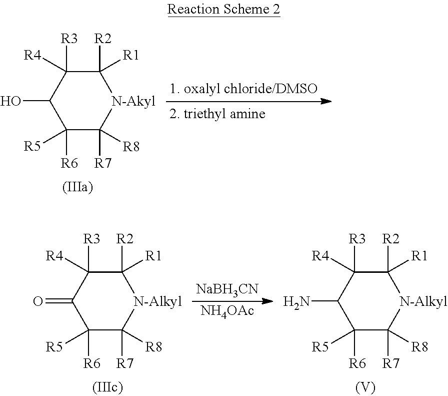

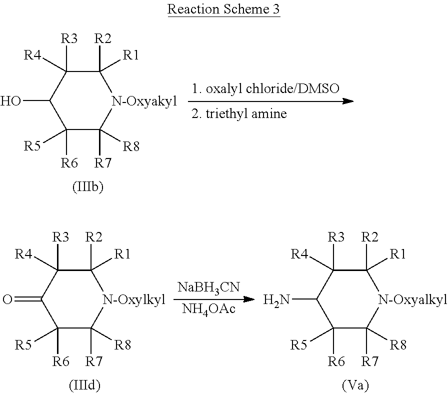

Conditions for Swern oxidation of alcohols to ketones are known to people of ordinary skill in the art, and have been disclosed, for example, in “Oxidation of alcohols by ‘activated’ dimethyl sulfoxide. A preparative, steric and mechanistic study”, Tetrahedron, 34 (11), 1978, (Omura et al.), and “Oxidation of alcohols by activated dimethyl sulfoxide and related reactions: An update”, Synthesis, (10); 857-70, (Tidwell et al.) Conditions for reductive amination of carbonyls with sodium cyannoborohydride are also known to people of ordinary skill in the art, and have been disclosed, for example, in “Reductive amination with sodium cyanoborohydride: N,N-dimethylcyclohexylamine”, Org. Synth. Coll., Vol. 6, 499, 1988, (Borch), and “Cyanohydriodoborate anion as a selective reducing agent”, J. Am. Chem. Soc., 95 (12), 1971, (Borch et al.)

As discussed above, one method to provide compounds of Formula (IV) or (IVa) wherein R′ is C₁ to C₄ alkyl is the use of a primary alkyl amine compound in the reductive amination reaction. As an alternative, compounds of Formulas (V) or (Va) can be alkylated by reaction of the primary amine with a compound of Formula (VI), as shown in Reaction Schemes 4 and 5. The resulting compounds wherein R′ is C₁ to C₄ alkyl are compounds of Formula (IVb) or (IVc). The chemical structure of compounds of Formula (IVb) and (IVc) is identical whether such compounds are made by reductive amination with a primary alkyl amine in a process similar to Reaction Scheme 2 or 3 or by alkylation as shown in Reaction Schemes 4 and 5.

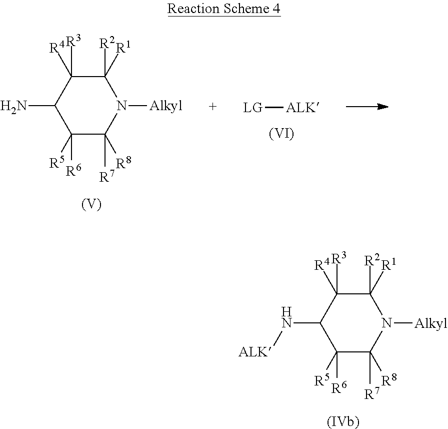

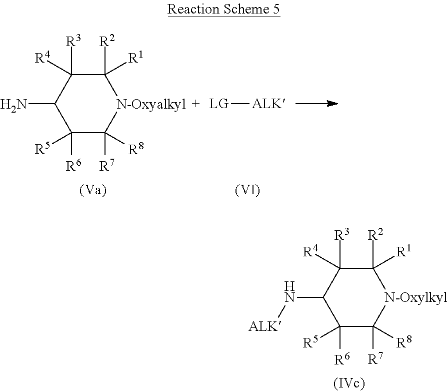

In the compound of Formula (VI), ALK′ is C₁ to C₄ alkyl and LG is a leaving group. Any suitable leaving group can be used, so long as the compound of Formula (VI) is reactive with the exocyclic amine of a compound of Formulas (V) or (Va). Suitable leaving groups include halide, such as chloride, bromide, and iodide, mesylate, tosylate, and the like. Likewise, ALK′ any suitable C₁ to C₄ alkyl can be used. Typical examples of C₁ to C₄ alkyl include methyl, ethyl, n-propyl, iso-propyl, and n-butyl. Methyl and ethyl are most common.

The ALK′ moiety in the compounds of Formulas (IVb) and (IVc) comes from the ALK′ group of compounds of Formula (VI), and is defined in the same way as that in compounds of Formula (VI).

The reaction shown in Reaction Schemes 4 and 5 can take place under any reaction conditions suitable for alkylation of a primary amine. Typically, the compound of Formula (V) or (Va) is first dissolved or dispersed in one or more inert diluents that do not undergo a chemical reaction under the alkylation conditions. Common inert diluents include aromatics such as benzene, toluene, and xylenes, ethers such as diethyl ether and tetrahydrofuran, as well as hydrocarbons such as hexanes. The compound of Formula (VI) can be added to the compound of Formula (V) or (Va) and the inert diluents in any suitable manner. For example, the compound of Formula (VI) can be added to the compound of Formula (V) or (Va) and the one or more inert diluents dropwise with a syringe. The reaction often takes place at ambient temperatures, but it can be facilitated by heating if necessary.

Compounds of Formulas (I), (II), and (IIa) can be formed by reacting a compound of Formula (III), including any compound of Formula (IIIa), (IIIa1), (IIIa2), (IIIb), (IIIb1), (IIIb2), or a compound of Formula (IV), including any compound of Formula (IVa), (IV1), (IV2), (IV3), (IVa1), (IVa2), or (IVa3), with an (alkyl)isocyanate compound having the structure (R^{b})ₘ—R^{a}—NCO. Some variations of this reaction are shown in Reaction Schemes 6, 7, 8, 9, and 10 which depict the reaction of such an (alkyl)isocyanate compound with compounds of Formulas (III), (IIIa), (IIIb), (IV), and (IVa) respectively.

In the isocyanate compound of structure (R^{b})ₘ—R^{a}—NCO, each R^{b} is an (alkyl)acryloyloxy group of Formula —OC(O)C(R^{d})═CH₂, which means that each group is an alkylacryloyloxy group (such as a methacryloyloxy group) or an acryloyloxy group. The alkylacryloyloxy group is typically methacrylolyoxy. The variable m refers to the number of (alkyl)acryloyloxy groups and can be any integer but is usually in a range of 1 to 6 or in a range of 1 to 3. In many embodiments, m is equal to 1 or 2. The group R^{a} is divalent radical (i.e., one R^{b} group), trivalent radical (i.e., two R^{b} groups), or higher polyvalent radical (i.e., 3 or more R^{b} groups). In many embodiments R^{a} is divalent or trivalent. If R^{a} is divalent, R^{a} is often an alkylene (i.e., an alkane-diyl). If R^{a} is trivalent, R^{a} is often an alkane-triyl.

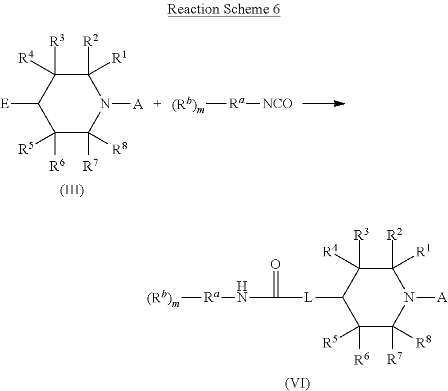

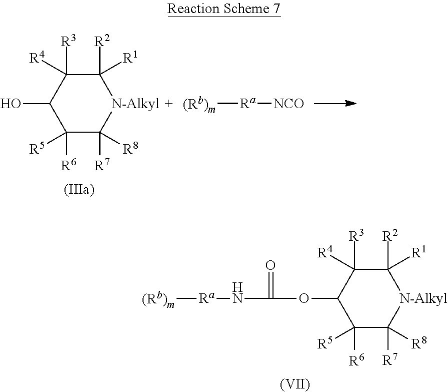

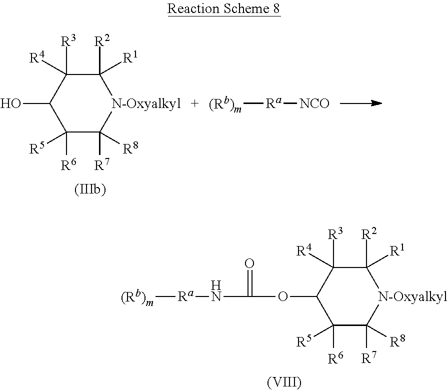

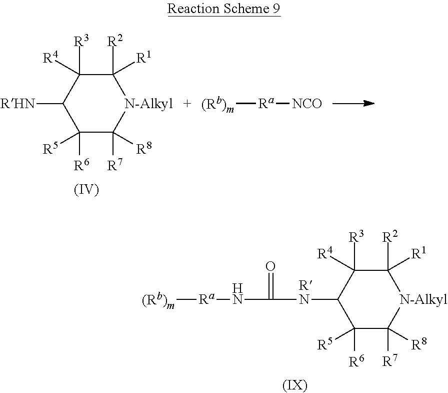

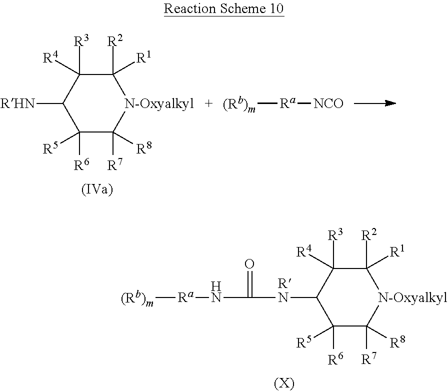

The reactions of Reaction Schemes 6, 7, 8, and 9 can take place under any conditions that are suitable for the condensation of an alcohol with an isocyanato group such as in the (alkyl)isocyanate compound depicted in the Reaction Schemes. Typically, the starting materials are dried, combined, and stirred until the reaction is complete. The progress of the reaction can be monitored by removing aliquots of the reaction mixture and analyzing the aliquots by Fourier transform infrared spectroscopy (FTIR). When FTIR no longer shows a prominent isocyanate absorption (typically at about 2250 cm⁻¹ to about 2275 cm⁻¹, such as at 2265 cm⁻¹), then the reaction is complete. The reaction can be carried out in one or more inert diluents that dissolve or disperse one or more of the starting materials or products, but do not undergo chemical reaction under the reaction conditions. Typical inert diluents include ethers such as diethyl ether and tetrahydrofuran, ketones such as acetone and methyl ethyl ketone, and aromatics such as benzene and toluene. If needed, the reaction mixture can be heated, for example to a temperature between 50° C. and 70° C., to facilitate the reaction. When the reaction is heated, it is often convenient to use an inert diluent that can reflux at the desired temperature in order to maintain that temperature. The reaction is often carried out under a dry atmosphere in order to minimize unwanted side reactions.

A catalyst can be used to accelerate the reaction depicted in Reaction Schemes 6-10. Suitable catalysts include, but are not limited to, amines and tin compounds. Examples of useful tin compounds include tin (II) and tin (IV) salts, such as stannous octoate, dibutyltin dilaurate, dibutyltin diacetate, dibutyltin di-2-ethylhexanoate, and dibutyltin oxide. Examples of useful amine compounds include tertiary amines such as triethylamine, tributylamine, triethylenediamine, tripropylamine, bis(dimethyllaminoethyl)ether, morphonilin compounds such as ethyl morpholine, and 2,2′-dimorpholinodiethyl ether, 1,4-diazobicyclo[2.2.2]oxtane abd 1m8-diazobicyclo[5.4.0]undec-7-ene (available from Aldrich Chemical Co., Milwaukee, Wis., USA, under the trade designations DABCO and DBU, respectively). In many cases, both a tin catalyst and an amine catalyst are used.

The catalyst, if employed, can be used in any suitable amount. Typically, the amount of tin catalyst is from about 50 to about 100,000 parts per million based on the total solids in the reaction, with a level of about 100 to 1,000 parts per million being most common. Amine catalysts are typically used at a level of about 0.001 to about 1 mol of amine per mol of the compound of Formula (III).

Catalysts such as those discussed above can be necessary to promote the reaction of a compound of Formula (III) with an isocyanate when E is OH. Thus, catalysts, usually tin catalysts such as those discussed above, are often used with the reaction of (IIIa) or (IIIb), that is, in Reaction Schemes 7 and 8. Amine catalysts and tin catalysts are often used together in such cases. In other cases, the catalyst may not be necessary to promote the reaction.

Many isocyanate compounds having the structure (R^{b})ₘ—R^{a}—NCO are commercially available, for example, from CBC America Corp. (Commack, USA) or Sigma-Aldrich (Milwaukee, USA). Such compounds include 2-isocyanatoethyl methacrylate, 2-isocyanatoethyl acrylate, 1,1-bis(acryloyloxymethyl)ethyl isocyanate, and the like.

The product of Reaction Scheme 6 is a compound of Formula (VI). In compounds of Formula (VI), R¹ through R⁸ and A are carried over from the compound of Formula (III) that is used in Reaction Scheme 6. Thus, R¹ through R⁸ and A have the same identity in compounds of Formula (VI) as in compounds of Formula (III). Likewise, R^{a}, R^{b}, and m are carried over from the isocyanate compound having the structure (R^{b})ₘ—R^{a}—NCO that is used in Reaction Scheme 6. The identity of R^{a}, R^{b}, and m are therefore the same as those in the compound having the structure (R^{b})ₘ—R^{a}—NCO that is used in Reaction Scheme 6. The identity of L in the compound of Formula (VI) depends on, but is not the same as, the identity of E in the compound of Formula (III) that is used in Reaction Scheme 6. When a compound of Formula (III) having an E that is OH is used in Reaction Scheme 6, then group L in the resulting compound of Formula (VI) is O. When a compound of Formula (III) having an E that is NR′H is used in Reaction Scheme 6, then L in the resulting compound of Formula (VI) is NR′. In such cases, the identity of R′ in the compound of Formula (VI) is identical to the identity of R′ in the compound of Formula (III) that is used in Reaction Scheme 6.

Reaction Schemes 7 and 8 depict the reaction of a compound of Formulas (IIIa) and (IIIb), respectfully, with a compound having the structure (R^{b})ₘ—R^{a}—NCO. The products of these reactions are compounds of Formulas (VII) and (VIII), respectfully. Compounds of Formula (VII) are compounds of Formula (VI) wherein L is O and A is alkyl. Thus, the identity of R¹ through R⁸, R^{a}, R^{b}, and m are as described above with respect to Formula (VI). Similarly, compounds of Formula (VIII) are compounds of Formula (VI) wherein L is O and A is oxyalkyl. In such compounds, the identity of R¹ through R⁸, R^{a}, R^{b}, and m are as described above with respect to Formula (VI).

Compounds of Formula (IX) are compounds of Formula (VI) wherein L is NR′ and A is alkyl. In such compounds, the identity of R¹ through R⁸, R^{a}, R^{b}, R′, and m are as described above with respect to Formula (VI). Similarly, compounds of Formula (X) are compounds of Formula (VI) wherein L is NR′ and A is alkyl. In such compounds, the identity of R¹ through R⁸, R^{a}, R^{b}, R′, and m are as described above with respect to Formula (VI).

Many compounds of Formula (VI) feature R³, R⁴, R⁵, and R⁶ that are H. Some compounds of Formula (VI) feature R¹, R², R⁷, and R⁸ that are C₁ to C₄ alkyl. Particular compounds of Formula (VI) feature R³, R⁴, R⁵, and R⁶ that are H and R¹, R², R⁷, and R⁸ that are C₁ to C₄ alkyl. In compounds of Formula (VI), R^{a} is commonly saturated hydrocarbon polyradical. C₁ to C₁₂ or C₁ to C₆ hydrocarbon polyradical is most common. When m is 1, then R^{a} is most often alkylene. C₁ to C₁₂ or C₁ to C₆ alkylene is most common. When m is 2, then R^{a} is often an alkane-triyl such as those having 1 to 12 carbon atoms, 2 to 10 carbon atoms, or 2 to 6 carbon atoms. R^{b} can be any (alkyl)acrylolyloxy, but is typically (meth)acrylolyloxy or acrylolyloxy.

Many compounds of Formula (VII) feature R³, R⁴, R⁵, and R⁶ that are H. Some compounds of Formula (VII) feature R¹, R², R⁷, and R⁸ that are C₁ to C₄ alkyl. Particular compounds of Formula (VII) feature R³, R⁴, R⁵, and R⁶ that are H and R¹, R², R⁷, and R⁸ that are C₁ to C₄ alkyl. In compounds of Formula (VII), R^{a} is commonly saturated hydrocarbon polyradical. C₁ to C₁₂ or C₁ to C₆ hydrocarbon polyradical is most common. When m is 1, then R^{a} is most often alkylene. C₁ to C₁₂ or C₁ to C₆ alkylene is most common. When m is 2, then R^{a} is often an alkane-triyl such as those having 1 to 12 carbon atoms, 2 to 10 carbon atoms, or 2 to 6 carbon atoms. Alkyl in compounds of Formula (VII) is most often C₁ to C₁₂ or C₁ to C₆ alkyl. Methyl is most common. R^{b} can be any (alkyl)acrylolyoxy, but is typically (meth)acrylolyoxy or acrylolyoxy.

Many compounds of Formula (VIII) feature R³, R⁴, R⁵, and R⁶ that are H. Some compounds of Formula (VIII) feature R¹, R², R⁷, and R⁸ that are C₁ to C₄ alkyl. Particular compounds of Formula (VIII) feature R³, R⁴, R⁵, and R⁶ that are H and R¹, R², R⁷, and R⁸ that are C₁ to C₄ alkyl. In compounds of Formula (VIII), R^{a} is commonly saturated hydrocarbon polyradical. C₁ to C₁₂ or C₁ to C₆ hydrocarbon polyradical is most common. When m is 1, then R^{a} is most often alkylene. C₁ to C₁₂ or C₁ to C₆ alkylene is most common. When m is 2, then R^{a} is often an alkane-triyl such as those having 1 to 12 carbon atoms, 2 to 10 carbon atoms, or 2 to 6 carbon atoms. The oxyalkyl in compounds of Formula (VIII) is most often C₁ to C₁₂ oxyalkyl. R^{b} can be any (alkyl)acrylolyloxy, but is typically (meth)acrylolyloxy or acrylolyloxy.

Many compounds of Formula (IX) feature R³, R⁴, R⁵, and R⁶ that are H. Some compounds of Formula (IX) feature R¹, R², R⁷, and R⁸ that are C₁ to C₄ alkyl. Particular compounds of Formula (IX) feature R³, R⁴, R⁵, and R⁶ that are H and R¹, R², R⁷, and R⁸ that are C₁ to C₄ alkyl. In compounds of Formula (IX), R^{a} is commonly saturated hydrocarbon polyradical. C₁ to C₁₂ or C₁ to C₆ hydrocarbon polyradical is most common. When m is 1, then R^{a} is most often alkylene. C₁ to C₁₂ or C₁ to C₆ alkylene is most common. When m is 2, then R^{a} is often an alkane-triyl such as those having 1 to 12 carbon atoms, 2 to 10 carbon atoms, or 2 to 6 carbon atoms. R′ in compounds of Formula (IX) is most often H. In some cases, R′ is C₁ to C₄ alkyl. In such cases, methyl and ethyl are often used, with methyl being most common. Alkyl in compounds of Formula (IX) is most often C₁ to C₁₂ or C₁ to C₆ alkyl. Methyl is most common. R^{b} can be any (alkyl)acrylolyoxy, but is typically (meth)acrylolyoxy or acrylolyoxy.

Many compounds of Formula (X) feature R³, R⁴, R⁵, and R⁶ that are H. Some compounds of Formula (X) feature R¹, R², R⁷, and R⁸ that are C₁ to C₄ alkyl. Particular compounds of Formula (X) feature R³, R⁴, R⁵, and R⁶ that are H and R¹, R², R⁷, and R⁸ that are C₁ to C₄ alkyl. In compounds of Formula (X), R^{a} is commonly saturated hydrocarbon polyradical. C₁ to C₁₂ or C₁ to C₆ hydrocarbon polyradical is most common. When m is 1, then R^{a} is most often alkylene. C₁ to C₁₂ or C₁ to C₆ alkylene is most common. When m is 2, then R^{a} is often an alkane-triyl such as those having 1 to 12 carbon atoms, 2 to 10 carbon atoms, or 2 to 6 carbon atoms. R′ in compounds of Formula (X) is most often H. In some cases, R′ is C₁ to C₄ alkyl. In such cases, methyl and ethyl are often used, with methyl being most common. The oxyalkyl in compounds of Formula (X) is most often C₁ to C₁₂ oxyalkyl. R^{b} can be any (alkyl)acrylolyoxy, but is typically (meth)acrylolyoxy or acrylolyoxy.

Exemplary compounds of Formulas (VI), (VII), (VIII), (IX), and (X) often feature R¹, R², R⁷, and R⁸ that are methyl, R³, R⁴, R⁵, and R⁶ that are H. In such cases, the compounds are compounds of Formulas (XI), (XII), (XIII), (XIV), and (XV), respectively.

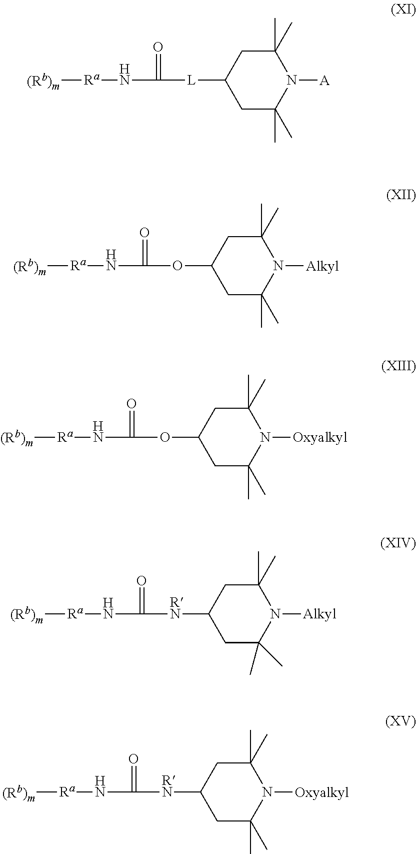

In compounds of Formula (XI), R^{a} is commonly saturated hydrocarbon polyradical, particularly when m is greater than 1. C₁ to C₁₂ or C₁ to C₆ hydrocarbon polyradical is most common. When m is 1, then R^{a} is most often alkylene. C₁ to C₁₂ or C₁ to C₆ alkylene is most common. R^{b} can be any (alkyl)acrylolyoxy, but is typically (meth)acrylolyoxy or acrylolyoxy.

In compounds of Formula (XII), R^{a} is commonly saturated hydrocarbon polyradical. C₁ to C₁₂ or C₁ to C₆ hydrocarbon polyradical is most common. When m is 1, then R^{a} is most often alkylene. C₁ to C₁₂ or C₁ to C₆ alkylene is most common. When m is 2, then R^{a} is often an alkane-triyl such as those having 1 to 12 carbon atoms, 2 to 10 carbon atoms, or 2 to 6 carbon atoms. Alkyl in compounds of Formula (VII) is most often C₁ to C₁₂ or C₁ to C₆ alkyl. Methyl is most common. R^{b} can be any (alkyl)acrylolyoxy, but is typically (meth)acrylolyoxy or acrylolyoxy.

In compounds of Formula (XIII), R^{a} is commonly saturated hydrocarbon polyradical. C₁ to C₁₂ or C₁ to C₆ hydrocarbon polyradical is most common. When m is 1, then R^{a} is most often alkylene. C₁ to C₁₂ or C₁ to C₆ alkylene is most common. When m is 2, then R^{a} is often an alkane-triyl such as those having 1 to 12 carbon atoms, 2 to 10 carbon atoms, or 2 to 6 carbon atoms. The oxyalkyl in compounds of Formula (VIII) is most often C₁ to C₁₂ oxyalkyl. Octyloxy is most common. R^{b} can be any (alkyl)acrylolyoxy, but is typically (meth)acrylolyoxy or acrylolyoxy.

In compounds of Formula (XIV), R^{a} is commonly saturated hydrocarbon polyradical. C₁ to C₁₂ or C₁ to C₆ hydrocarbon polyradical is most common. When m is 1, then R^{a} is most often alkylene. C₁ to C₁₂ or C₁ to C₆ alkylene is most common. When m is 2, then R^{a} is often an alkane-triyl such as those having 1 to 12 carbon atoms, 2 to 10 carbon atoms, or 2 to 6 carbon atoms. R′ in compounds of Formula (IX) is most often H. In some cases, R′ is C₁ to C₄ alkyl. In such cases, methyl and ethyl are often used, with methyl being most common. Alkyl in compounds of Formula (IX) is most often C₁ to C₁₂ or C₁ to C₆ alkyl. Methyl is most common. R^{b} can be any (alkyl)acrylolyoxy, but is typically (meth)acrylolyoxy or acrylolyoxy.

In compounds of Formula (XV), R^{a} is commonly saturated hydrocarbon polyradical. C₁ to C₁₂ or C₁ to C₆ hydrocarbon polyradical is most common. When m is 1, then R^{a} is most often alkylene. C₁ to C₁₂ or C₁ to C₆ alkylene is most common. When m is 2, then R^{a} is often an alkane-triyl such as those having 1 to 12 carbon atoms, 2 to 10 carbon atoms, or 2 to 6 carbon atoms. R′ in compounds of Formula (X) is most often H. In some cases, R′ is C₁ to C₄ alkyl. In such cases, methyl and ethyl are often used, with methyl being most common. The oxyalkyl in compounds of Formula (X) is most often C₁ to C₁₂ oxyalkyl. Octyloxy is most common. R^{b} can be any (alkyl)acrylolyoxy, but is typically (meth)acrylolyoxy or acrylolyoxy.

The compounds discussed herein contain (alkyl)acrylolyoxy groups, and can be incorporated into the backbone of one or more polymers or copolymers. Any polymer or copolymer can be used, so long as the method used to polymerize the monomeric or co-monomeric components of the polymer or copolymer can polymerize the (alkyl)acrylolyloxy-containing compounds discussed herein.

(Alkyl)acrylolyoxy-containing compounds, such as those disclosed herein, are typically polymerized by radical polymerization processes. As such, when the compounds discussed herein are incorporated into polymers or copolymers, the monomers or co-monomers used are often radically polymerizable. Examples of radically polymerizable monomers and co-monomers include styrene, (alkyl)acrylates, acrylamides, vinyl chloride, vinyl fluoride, vinylidene chloride, vinylidene fluoride, and the like. (Alkyl)acrylates, which are typically (meth)acrylates, such as methacrylates or acrylates, are most common.

Exemplary radically polymerizable monomers and co-monomers that can be used as monomers or co-monomers include methyl (meth)acrylate, ethyl acrylate, isopropyl methacrylate, n-hexyl acrylate, stearyl acrylate, allyl acrylate, glycerol triacrylate, ethyleneglycol diacrylate, diethyleneglycol diacrylate, triethyleneglycol dimethacrylate, 1,3-propanediol di(meth)acrylate, trimethylolpropane triacrylate, 1,2,4-butanetriol trimethacrylate, 1,4-cyclohexanediol diacrylate, pentaerythritol tetra(meth)acrylate, sorbitol hexacrylate, tetrahydrofurfuryl (meth)acrylate, bis[1-(2-acryloxy)]-p-ethoxyphenyldimethylmethane, bis[1-(3-acryloxy-2-hydroxy)]-p-propoxyphenyldimethylmethane, ethoxylated bisphenolA di(meth)acrylate, and tris-hydroxyethyl-isocyanurate trimethacrylate; (meth)acrylamides (i.e., acrylamides and methacrylamides) such as (meth)acrylamide, methylene bis-(meth)acrylamide, and diacetone (meth)acrylamide; urethane (meth)acrylates; the bis-(meth)acrylates of polyethylene glycols (preferably of molecular weight 200-500), copolymerizable mixtures of acrylated monomers such as those in U.S. Pat. No. 4,652,274 (Boettcher et al.), acrylated oligomers such as those of U.S. Pat. No. 4,642,126 (Zador et al.), and poly(ethylenically unsaturated) carbamoyl isocyanurates such as those disclosed in U.S. Pat. No. 4,648,843 (Mitra); and vinyl compounds such as styrene, diallyl phthalate, divinyl succinate, divinyl adipate and divinyl phthalate. Siloxane-functional (meth)acrylates as disclosed, for example, in WO-00/38619 (Guggenberger et al.), WO-01/92271 (Weinmann et al.), WO-01/07444 (Guggenberger et al.), WO-00/42092 (Guggenberger et al.) and fluoropolymer-functional (meth)acrylates as disclosed, for example, in U.S. Pat. No. 5,076,844 (Fock et al.), U.S. Pat. No. 4,356,296 (Griffith et al.), EP-0373 384 (Wagenknecht et al.), EP-0201 031 (Reiners et al.), and EP-0201 778 (Reiners et al.) can also be used.

The resulting polymer or copolymer can be used in the same manner as the parent polymer or copolymer (that is, the corresponding polymer or copolymer that does not have a compound described herein incorporated therein). The hindered amine light stabilizer compounds disclosed herein, when incorporated into a polymer or copolymer as described, can provide a polymer or copolymer that is resistant to the adverse effects of actinic radiation, such as visible and ultraviolet light.

Articles can comprise one or more of the compounds, polymers, or copolymers discussed herein. Such articles can also be resistant to the adverse effects of actinic radiation, such as visible and ultraviolet light.

### List of Exemplary Embodiments

The following list of embodiments is intended to better illustrate particular aspects of the disclosure. None of the embodiments enumerated below are intended to be limiting, unless otherwise specified.

Embodiment 1 is a compound can have the structure of Formula (1):

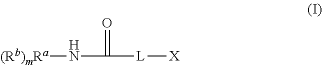

wherein:

X is

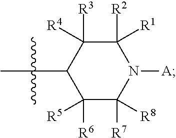

R¹ is alkyl;

R² is alkyl;

R³ is H or alkyl;

R⁴ is H or alkyl;

R⁵ is H or alkyl;

R⁶ is H or alkyl;

R⁷ is alkyl; and

R⁸ is alkyl;

A is alkyl or oxyalkyl;

L is O or NR′;

R′ is H or C₁ to C₄ alkyl;

m is 1-6;

R^{a} is a connecting group having a valence of m+1; and

R^{b} is an (alkyl)acrylolyoxy group functional group of the formula OC(O)C(R^{d})═CH₂ wherein R^{d} is alkyl or H.

Embodiment 2 is a compound of embodiment 1 having a structure of Formula (II)

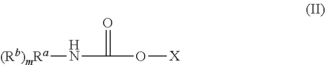

or having a structure of Formula (IIa)

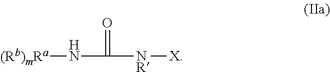

Embodiment 3 is a compound of any of the preceding embodiments wherein m is 1 or 2.

Embodiment 4 is a compound of any of the preceding embodiments wherein m is 1.

Embodiment 5 is a compound of any of the preceding embodiments wherein m is 2.

Embodiment 6 is a compound of any of the preceding embodiments wherein R^{a} is alkylene.

Embodiment 7 is a compound of embodiment 6 wherein the alkylene is C₁ to C₆ alkylene.

Embodiment 8 is a compound of any of embodiments 1-6 or 7 wherein R^{a} is hydrocarbon polyradical.

Embodiment 9 is a compound of embodiment 8 wherein the hydrocarbon polyradical is C₁ to C₆ hydrocarbon polyradical. The hydrocarbon polyradical is often an alkane-diyl or alkane-triyl.

Embodiment 10 is a compound of any of the preceding embodiments wherein A is alkyl.

Embodiment 11 is a compound of embodiment 10 wherein A is C₁ to C₁₂ alkyl.

Embodiment 12 is a compound of embodiment 11 wherein A is C₁ to C₆ alkyl.

Embodiment 13 is a compound of embodiment 12 wherein A is methyl.

Embodiment 14 is a compound of any of embodiments 1-9 wherein A is oxyalkyl.

Embodiment 15 is a compound of embodiment 14 wherein A is C₁ to C₁₂ oxyalkyl.

Embodiment 16 is a compound of embodiment 15 wherein A is C₈ oxyalkyl.

Embodiment 17 is a compound of any of the preceding embodiments wherein R^{b} is OC(O)C(R^{d})═CH₂ and R^{d} is H.

Embodiment 18 is a compound of any of embodiments 1-16 wherein R^{b} is OC(O)C(R^{d})═CH₂ and R^{d} is alkyl.

Embodiment 19 is a compound of embodiment 18 wherein the alkyl R^{d} group is C₁ to C₁₂ alkyl.

Embodiment 20 is a compound of embodiment 19 wherein the alkyl R^{d} group is C₁ to C₆ alkyl.

Embodiment 20a is a compound of embodiment 20 wherein the alkyl R^{d} group is methyl.

Embodiment 21 is a compound of any of embodiments 1-16 wherein R^{b} is OC(O)C(R^{d})═CH₂ and R^{d} is H or methyl.

Embodiment 22 is a compound of any of the preceding embodiments wherein R¹ is C₁ to C₆ alkyl.

Embodiment 23 is a compound of embodiment 10 wherein R¹ is methyl.

Embodiment 24 is a compound of any of the preceding embodiments wherein R² is C₁ to C₆ alkyl.

Embodiment 25 is a compound of embodiment 24 wherein R² is methyl.

Embodiment 26 is a compound of any of the preceding embodiments wherein R³ is H.

Embodiment 27 is a compound of any of the preceding embodiments wherein R⁴ is H.

Embodiment 28 is a compound of any of the preceding embodiments wherein R⁵ is H.

Embodiment 29 is a compound of any of the preceding embodiments wherein R⁶ is H.

Embodiment 30 is a compound of any of the preceding embodiments wherein R⁷ is C₁ to C₆ alkyl.

Embodiment 31 is a compound of embodiment 30 wherein R⁷ is methyl.

Embodiment 32 is a compound of any of the preceding embodiments wherein R⁸ is C₁ to C₆ alkyl.

Embodiment 33 is a compound of any of embodiment 32 wherein R⁸ is methyl.

Embodiment 34 is a compound of any of the preceding embodiments wherein the compound is a compound of Formula (VI).

Embodiment 35 is a compound of any of embodiments 1-33 wherein the compound is a compound of Formula (VII).

Embodiment 36 is a compound of any of embodiments 1-33 wherein the compound is a compound of Formula (VIII).

Embodiment 37 is a compound of any of embodiments 1-33 wherein the compound is a compound of Formula (IX).

Embodiment 38 is a compound of any of embodiments 1-33 wherein the compound is a compound of Formula (X).

Embodiment 39 is a compound of any of embodiments 1-33 wherein the compound is a compound of Formula (XI).

Embodiment 40 is a compound of any of embodiments 1-33 wherein the compound is a compound of Formula (XII).

Embodiment 41 is a compound of any of embodiments 1-33 wherein the compound is a compound of Formula (XIII).

Embodiment 42 is a compound of any of embodiments 1-33 wherein the compound is a compound of Formula (XIV).

Embodiment 43 is a compound of any of embodiments 1-33 wherein the compound is a compound of Formula (XV).

Embodiment 44 is a polymer or copolymer derived from a compound of any of the preceding embodiments.

Embodiment 45 is a polymer or copolymer or embodiment 44, wherein the polymer or copolymer is a copolymer that is also derived from at least one comonomer.

Embodiment 46 is a copolymer of embodiment 45 wherein the at least one comonomer comprises an acrylic comonomer or an acrylate comonomer.

Embodiment 47 is a copolymer of embodiment 46 wherein the acrylate comonomer comprises at least one (meth)acrylate.

Embodiment 48 is a copolymer or embodiment 47 wherein the at least one (meth)acrylate is methyl methacrylate.

Embodiment 49 is a copolymer of embodiment 46 wherein the acrylic comonomer comprises acrylic acid.

Embodiment 50 is a copolymer of embodiment 45 wherein the at least one comonomer comprises styrene.

Embodiment 51 is an article comprising a compound of any of embodiments 1-43 or a polymer or copolymer of any of embodiment 44-50.

Embodiment 52 is an article of embodiment 51, wherein the article is a coated article. That is, the article comprises a substrate and a coating comprising a compound of any embodiment 1-43 or a polymer or copolymer of any of embodiment 44-51 on a surface of the substrate.

Embodiment 53 is an article of any of embodiments 51-52, wherein the article is a molded article.

### EXAMPLES

Materials 1,2,2,6,6-pentamethyl-4-hydroxy-piperidine (PMHP) was obtained from TCI America, Portland, Oreg.

TINUVIN 123, IRGACURE 184, and IRGACURE 819 were obtained from BASF Florham Park, N.J., under trade designations “TINUVIN 123”, “IRGACURE 184”, and “IRGACURE 819” respectively.

1,1-bis(acryloyloxymethyl) ethyl isocyanate (BEI), isocyanatoethyl acrylate (AOI), and isocyanatoethyl methacrylate (MOI), were obtained from obtained CBC America Corp., Commack, N.Y.

Bis-(3-trimethoxysilylpropyl)amine was obtained from Evonik, Piscataway, N.J., under trade designation “DYNASLAN 1124”, or alternatively, from Momentive, Huntersville, N.C., as “SILQUEST 1170”.

Tetrahydrofuran (THF), methyl ethyl ketone (MEK), methyl t-butyl ether (MTBE), sodium carbonate, sodium hydroxide, anhydrous magnesium sulfate, 85% potassium hydroxide, dimethylsulfoxide (DMSO), methylene chloride (dichloromethane), methanol, chloroform, and triethyl amine were obtained from EMD Chemicals, Gibbstown, N.J.

Hydroxyethyl acrylate (HEA), 4-methoxy phenol (MEHQ), triethylamine, dibutyltindilaurate (DBTDL), acryloyl chloride, oxalyl chloride, and sodium cyanoborohydride were obtained from Sigma-Aldrich, Milwaukee, Wis.

Ammonium acetate was obtained from VWR, West Chester, Pa.

EBECRYL 600, epoxy acrylate of the diglycidyl ether of bisphenol A, was obtained from Allnex, Alpharetta, Ga., under trade designation “EBECRYL 600”.

Pentaerythritol triacrylate (PET3A) was obtained from Sartomer Company of Exton, Pa., under the designation “SR444C”.

Hexanediol diacrylate was obtained from Sartomer Company of Exton, Pa., under the designation “SR238”.

1-methoxy-2,2,5,5-tetramethylpiperidin-4-ol was prepared using a procedure reported by Schoening et al., (J. Org. Chem., 2009, 74, 1567-1573).

### Preparative Example 1

### Preparation of a Compound of Formula (IIIb2)

A 1 L 3-necked round bottom equipped with overhead stirrer and a vacuum bearing was charged with 200 g (0.275 mol, 0.55 eq, 737 MW) TINUVIN 123, and 323 g ethanol and placed in an oil bath at 70° C. To the reaction was added 73.23 g (1.109 mol, 66.01 MW) 85% potassium hydroxide. As the base was added the color of the reaction mixture changed from yellow to orange to brown; the reaction mixture also began refluxing. The bottom of the flask was scraped to provide a homogeneous mixture.

After the reaction mixture refluxed for 3.5 hours, the flask was fitted with a distillation head and condenser and placed under aspirator vacuum. 215 g of ethanol was collected by distillation, after which the reaction mixture was a thick, taffy-like mass. 250 g of water was added to the reaction mixture and the inside of the flask was scraped to disperse or dissolve the solids. The mixture was stirred for about 10 min at about 50° C., after which 300 g MTBE was added to the flask and the mixture was stirred for an additional 10 min. The reaction mixture was then poured into a 2 L separatory funnel, the bottom layer drained off and the top layer washed with 250 g water in the funnel. After removing the aqueous layer, the organic layer was dried over anhydrous magnesium sulfate, filtered, and concentrated on a rotary evaporator under aspirator pressure at 90° C. for 2 h to provide 137.2 g (87%) of undistilled product. This was distilled at 140° C. (pot temperature) at 29.3 Pa to provide 127.5 g (80.8%) of product.

### Preparative Example 2

### Preparation of a Ketone Intermediate

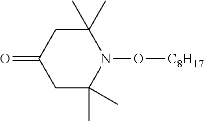

To a 500 mL 3-neck flask equipped with an overhead stirrer and nitrogen inlet adapter, and rubber septum was charged 12.04 g (0.1541 mol) dimethyl sulfoxide and 226 g of methylene chloride. The reaction was put under a nitrogen atmosphere and placed in an isopropanol dry-ice bath. After a few minutes, 9.78 g (0.0770 mol) oxalyl chloride was added via syringe through the septum over one minute. Five minutes later 20.00 g (0.0701 mol, approximate molecular weight 285.47 g/mol) 2,2,6,6-tetramethyl-4-hydroxy-1-octyloxy-piperidine (the product of Preparative Example 1) was slowly added by syringe through the septum over 15 minutes. After stirring for an additional 15 minutes, 17.72 g (0.17515 mol) triethylamine was added by syringe over about 30 seconds. Stirring was continued for 10 minutes in the isopropanol and dry ice bath, followed by stirring for 10 minutes at room temperature. The resulting solution was washed with 333 mL of 2-N hydrochloric acid, providing a mixture with distinct organic and aqueous layers. The organic and aqueous layers were separated, and the aqueous layer was extracted with 200 g of chloroform. The chloroform was combined with the other organic layer, and combined organic layers were dried over anhydrous magnesium sulfate, filtered, and concentrated on a rotary evaporator under water aspirator pressure at about 65° C. for 2 hours to provide an oil. The product was evaluated by ¹H NMR and FTIR, which gave results consistent with the expected structure.

### Preparative Example 3

### Preparation of the Compound of Formula (IVa3)

A 250 mL 3-necked flask equipped with overhead stirrer was charged with 5.00 g (0.017639 mol) 2,2,6,6-tetramethyl-4-keto-1-octyloxy-piperidine (the product of Preparative Example 2,) 8 g of 3 angstrom molecular sieves, 13.60 g (0.17639 mol) ammonium acetate, and 77.5 g methanol and stirred for 1.75 hours under nitrogen at room temperature, after which 1.51 g (0.0242 mol) sodium cyanoborohydride in 13 g methanol was added to the reaction over 45 minutes and allowed to stir overnight. 360 g chloroform was then added to the reaction mixture and the mixture was washed twice with 400 g of 1N sodium hydroxide, dried over anhydrous magnesium sulfate, filtered, and concentrated at 40° C. at aspirator pressure on a rotary evaporator. Analysis by ¹H NMR showed the reaction to be a mixture of about 70 mole percent of the desired amine, 18 mole percent of a secondary amine, and 12 mole percent of the starting material. The products were separated to flash chromatography using an Analogix Intelliflash 280 from Agilent Technologies, Inc., Santa Clara, Calif. with a 150 g, 40 mm diameter column using a gradient of 25-30% methanol in methylene chloride over 20 minutes and then 30% methanol in methylene chloride to provide the desired product (2,2,6,6-tetramethyl-4-amino-1-octyloxy-piperidine) as an oil.

### Example 1

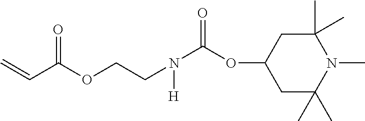 [SMILES: C=CC(=O)OCCNC(=O)OC1CC(C)(C)N(C)C(C)(C)C1]

A 500 mL round bottom flask with a stir bar was charged with 49.97 g PMHP and about 300 g of MEK. The flask was placed in an oil bath, fitted with a distillation head, condenser, and receiver under nitrogen. The temperature of the bath was raised to about 95° C., and about half of the MEK was removed to azeotropically dry the contents. The round bottom flask was then placed on a rotary evaporator and concentrated at aspirator pressure at about 95° C. for 1 hour to provide 49.85 g (0.291 mol) of dried, molten PMHP. Into the flask of molten PMHP was added 213 microliters of DBTDL (2,000 ppm) and 41.11 g (0.291 mol) isocyanatoethyl acrylate. The flask was placed in a 55° C. oil bath under dry air and allowed to stir for one hour, after which FTIR analysis of the reaction showed no isocyanate absorption at 2265cm⁻¹. The product was isolated a solid after cooling to ambient temperature.

### Example 2

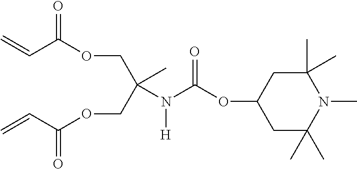 [SMILES: C=CC(=O)OCC(C)(COC(=O)C=C)NC(=O)OC1CC(C)(C)N(C)C(C)(C)C1]

A three-necked 500 mL round bottom flask equipped with stir bar was charged with 44.18 g (0.2579 mol) PHMP, which was dried according the procedure discussed above in Example 1. The flask was then placed in a 75° C. oil bath under dry air. When the PHMP fully molted, the flask was charged with 633 microliters DBTDL (2,000 ppm). Using an addition funnel, 61.71 g (0.2579 mol) BEI was charged into the reaction over 20 minutes. After 30 minutes, FTIR analysis of the reaction showed no isocyanate absorption at 2265 cm⁻¹, and the product was obtained an oil.

### Example 3

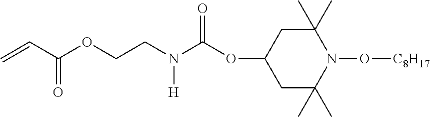

A 500 mL round bottom was charged with 150 g (0.525 mol) 2,2,6,6-tetramethyl-4-hydroxy-1-octyloxy-piperidine (the product of Preparative Example 1), 77.86 g (0.552 mol) IEA, 53.17 g (0.525 mol) triethylamine, and 2.7 mL of a 10% solution of DBTDL in MEK (about 1,000 ppm DBTDL). The reaction mixture was heated to 75° C. with stirring under dry air for about 2 hours, at which time FTIR analysis showed no isocyanate absorption. The reaction was then concentrated on a rotary evaporator at 90° C. under aspirator pressure to remove solvents and provide the desired product.

### Comparative Example 1

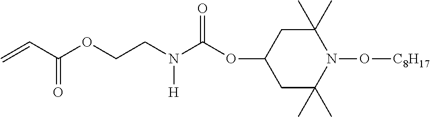

### Attempted Preparation of Without Amine Catalyst.

In a manner similar to Example 3, 50.04 g (0.175 mol, 285.47 MW) 2,2,6,6-tetramethyl-4-hydroxy-1-octyloxy-piperidine (the product of Preparative Example 1), 24.74 g (0.175 mol, 141.12 MW) IEA, and 440 microliters DBTDL in MEK (˜2350 ppm DBTDL), and heated at 75° C. for about 2 h under dry air, at which time FTIR analysis showed no NCO peak. ¹H NMR analysis shows about 24% of the starting alcohol compared to product, on a molar basis, indicating that the reaction did not go to completion.

### Example 4

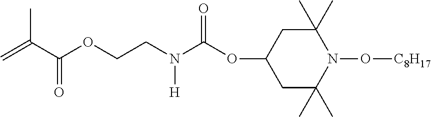

A procedure analogous to that of Example 3 was followed, except that 50.04 g (0.175 mol) of 2,2,6,6-tetramethyl-4-hydroxy-1-octyloxy-piperidine (the product of Preparative Example 1), 24.74 g (0.175 mol) of IEA, and 440 microliters of DBTL in MEK were used in the reaction mixture.

### Example 5

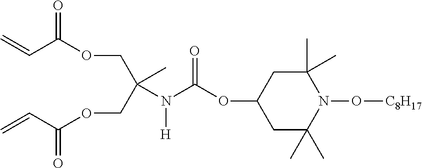

A procedure analogous to that of Example 3 was followed, except that 24.98 g (0.0875 mol) 2,2,6,6-tetramethyl-4-hydroxy-1-octyloxy-piperidine (the product of Preparative Example 1,) 22.44 g (0.0937 mol) BEI, 8.86 g (0.0875 mol) triethylamine, and 270 microliters of DBTDL in MEK were used in the reaction mixture.

### Example 6

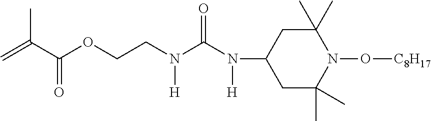

A vial was charged with 0.50 g (0.0003223 mol) isocyanatoethyl methacrylate, 1.16 g methylene chloride, and 6 microliters of 10% DBTDL in methylene chloride (about 5,000 ppm based on total solids). A solution of 0.92 g (0.0003223 mol) of the product of Preparative Example 3 in 2.14 methylene chloride was added to the vial over 2 minutes. After 25 min, 0.023 g more of the product of Preparative Example 3 was added to the reaction. After a total of 30 minutes of reaction, FTIR analysis showed no isocyanate absorption. The material was concentrated on a rotary evaporator to provide a thick oil. ¹H NMR analysis confirmed the identity of the desired product.

### Example 7

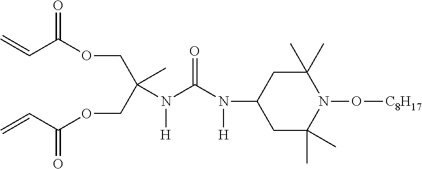

A vial was charged with 0.50 g (0.00209 mol) BEI, 2.0 g methylene chloride and 6 microliters 10% DBTDL in methylene chloride (about 5,000 ppm based on total solids) and placed in an ice water bath. A solution of 0.59 g (0.00209 mol) of the product of Preparative Example 3 in 3.26 g methylene chloride was added to the vial over about 7 minutes. After 23 minutes, 0.010 g more amine was added to the reaction. After a total of 43 minutes of reaction time, FTIR analysis showed no isocyanate absorption. The material was concentrated on a rotary evaporator to provide a thick oil. ¹H NMR analysis confirmed the identity of the desired product.

### Example 8

A stock solution was prepared from 40 g EBECRYL 600, 10 g SR238, 1.0 g IRGACURE 184, 0.5 g IRGACURE 819, 1.0 g of a 10% solution of TEGORAD 2100 in MEK, and 77.25 g MEK by mixing the ingredients and agitating the mixture until a homogeneous solution was formed.

A first solution (Solution A) was prepared from 30 g of the stock solution and 1.17 g of a 10% (by solids content) solution of the product of Example 3 in MEK.

A second solution (Solution B) was prepared from 30 g of the stock solution, 1.17 g of a 10% by solids solution of acrylated benzotriazole (CAS number 96478-09-0) in MEK, and 1.17 g of a 10% (by solids content) solution of the product of Example 4 in MEK.

Each solution was separately coated onto a 4 mm primer PET slide (available from E. I. du Pont de Nemours and Co. (Wilmington, Del., USA) under the trade designation MELINEX 618) using a #10 wire wound bar and dried for 2 minutes at 80° C. The dried coatings had a thickness of about 6 micrometers.

The coatings were placed under a nitrogen atmosphere and cured with an HP-6 High Power Six-Inch UV Lamp system with a VPS-3 Power Supply using a 300 W/in Fusion H-type bulb (all components commercially available from Heraeus Noblelight Fusion UV Inc., Gaithersburg, USA) operating at 100% power at a 30 feet/minute (9.14 minute) setting.

1. A compound can have the structure of Formula (II): 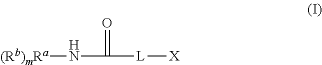
wherein: 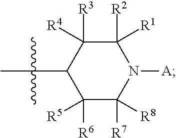
X is
R¹ is alkyl;
R² is alkyl;
R³ is H or alkyl;
R⁴ is H or alkyl;
R⁵ is H or alkyl;
R⁶ is H or alkyl;
R⁷ is alkyl;
R⁸ is alkyl;
A is alkyl or oxyalkyl;
L is O or NR′;
R′ is H or C₁ to C₄ alkyl;
m is 2-6;
R^{a} is a connecting group having a valence of m+1; and
R^{b} is an (alkyl)acrylolyoxy group functional group of the formula OC(O)C(R^{d})═CH₂, wherein R^{d} is alkyl or H.

2. The compound of claim 1 wherein m is 2.

3. The compound of any of claim 1, wherein R^{a} is alkylene or a hydrocarbon polyradical.

4. The compound of claim 3 wherein the hydrocarbon polyradical is trivalent.

5. The compound of claim 3, wherein the hydrocarbon polyradical is C₁ to C₆ hydrocarbon polyradical.

6. The compound of claim 1 wherein A is C₁ to C₁₂ alkyl.

7. The compound of claim 1 wherein A is C₁ to C₁₂ oxyalkyl.

8. The compound of claim 1 wherein each of R¹, R², R⁷ and R⁸ is alkyl.

9. The compound of claim 8, wherein the alkyl is methyl.

10. The compound of claim 1, wherein each of R³, R⁴, R⁵, and R⁶ is H.

11. The compound of claim 1, wherein R^{b} is OC(O)C(R^{d})═CH₂ and R^{d} is H or methyl.

12. The compound of claim 1, wherein R^{b} is OC(O)C(R^{d})═CH₂ and R^{d} is C₁ to C₆ alkyl.

13. A polymer or copolymer derived from a compound of claim 1.

14. A polymer or copolymer of claim 13, wherein the polymer or copolymer is a copolymer that is also derived from one or more comonomers.

15. An article comprising a polymer or copolymer of claim 13.
